SEQUENCE LISTING (5 sequences)

SEQ ID NO: 1 - DNA, 30 bp, Artificial Sequence
  Description of Artificial Sequence Synthetic oligonucleotide
cgcttgagatctaattcgtcgaccatggaa

SEQ ID NO: 2 - DNA, 30 bp, Artificial Sequence
  Description of Artificial Sequence Synthetic oligonucleotide
agacaagcggccgcaagttaccccctaatg

SEQ ID NO: 3 - DNA, 19 bp, Artificial Sequence
  Description of Artificial Sequence Synthetic oligonucleotide
atgatgttaactggggacc

SEQ ID NO: 4 - DNA, 35 bp, Artificial Sequence
  Description of Artificial Sequence Synthetic oligonucleotide
taatgtgcagagcttaaactgttgctgctgctact

SEQ ID NO: 5 - DNA, 2460 bp, Artificial Sequence
  Description of Artificial Sequence Synthetic polynucleotide
atggaacctgcaactcttactcgttcttcctctcttactagattcccttatcgtcgtggtttatccactctccgactcgctcgagtcaactcgttctcaattcttccacctaaaactcttctccgtcaaaaaccgcttcgtatctctgcttcccttagtcttccaccacggtcgattcgtctacgtgctgtcgaagatcaccatcacgatcatcatcacgatgacgagcaagatcatcacaaccaccatcatcatcaccatcaacacggatgctgttctgtggaattgaaagcggagagtaagcctcagaaggtgttgttcggattcgctaaagctatcggatgggttagattggccaattacctcagagagcatcttcatctttgctgctccgccgctgcaatgttcctcgctgccgccgtctgtccttaccttgctcctgaaccttacattaagtctcttcagaacgcattcatgattgttggttttcctcttgttggagtttcagcatctctcgacgcacttatggatatagctggaggaaaagtgaacatccatgtcttgatggcacttgcggcttttgcatctgtgtttatgggaaatgctttggaaggaggattgcttctagctatgttcaatcttgctcatattgctgaggagttctttactagtcgatcaatggtggatgtcaaagaattgaaagagagtaatccagattctgcattgttgatcgaagtacacaatggcaatgttccaaatatatctgatttgtcatacaaaagcgttcctgtgcacagcgtagaagttggatcctatgttttggttggaactggtgagattgtgcctgtagattgcgaagtctatcaaggtagtgctacaattacaattgagcacttgactggggaagtcaagccgttggaggcaaaagctggagatagagtgcctggtggtgcaagaaatttggatggcagaatgattgtaaaggctacaaaggcatggaatgattcgacgcttaacaagattgtacagctgaccgaggaagcacattctaataaacccaaacttcagagatggctggatgagtttggcgagaattacagcaaggttgtcgttgttttgtcacttgcaattgccttcctaggtccatttttgttcaagtggccttttctcagcaccgcagcatgtagaggatctgtttacagagcattgggacttatggtggccgcatcaccatgtgctctggccgtagctccattggcttatgctactgctattagttcctgtgcaagaaagggaatattgctgaaaggtgcacaggttctagatgctcttgcgtcttgccatactattgcttttgacaaaactggtaccttaacaaccggcggccttacttgtaaagcaattgaacccatttatgggcaccaaggaggaactaattcaagtgtaataacttgctgcattccaaattgtgaaaaagaagctcttgcagttgcggctgccatggagaagggcaccacgcatcctattggaagagctgttgtagatcacagtgtgggtaaggatcttccttctatttttgttgaaagcttcgaatattttcctggtagaggccttactgctactgtcaacggtgttaagacagtagctgaagagagtagattacgaaaagcatcacttggttctatagagttcattacctcacttttcaaatctgaagatgaatctaaacagatcaaggatgctgtaaacgcgtcttcgtacggaaaggacttcgttcatgctgctctttctgttgatcaaaaggtaacattgattcacctcgaagatcagcctcgtccaggggtgtcaggagttatagcagaacttaaaagctgggccagactccgagtaatgatgttaactggggaccatgattcaagtgcttggagagttgcaaacgcagtgggtattaccgaagtctactgcaacctaaagtcagaggataagttaaatcatgtaaagaacattgctcgggaagcaggtggaggtttaattatggtaggagaagggattaatgatgctccagctctagcagctgcaacagtggggattgttcttgctcaacgagcgagtgccactgcgattgccgttgctgacatcttactgcttcgagacaacatcaccggtgttccgttctgtgtcgctaaatcccgccagacaacatcattggtcaagcaaaacgtagctcttgcattaacatcgatattcttggccgctcttccttcagttttagggtttgtcccattgtggttgacggtacttctacatgaaggcgggactcttctggtgtgtctaaactcagtacgtggtctaaacgatccatcatggtcgtggaaacaagacatagttcatctaatcaacaagttacgctcacaagaaccaaccagtagcagcagcaacagtttaagctctgcacattag

(19) United States
(12) Patent Application Publication
(10) Pub. No.: US 2011/0223649 A1
(43) Pub. Date: Sep. 15, 2011

(54) RECOMBINANT CELL TRANSFORMED BY AN ARABIDOPSIS THALIANA ATPASE ATHMA1 SEQUENCE

(76) Inventor: Viviana Rosa Ordenes Ortiz, La Serena (CL)

(21) Appl. No.: 12/992,457

(22) PCT Filed: May 14, 2009
(86) PCT No.: PCT/EP2009/055823

§ 371 Date: May 24, 2011

(30) Foreign Application Priority Data
May 15, 2008 (CL) 14242008

(51) Int. Cl. C12N 1/21; C12N 1/19; C02F 3/34

(52) U.S. Cl. 435/252.3; 435/254.2; 435/262

(57) ABSTRACT

This invention corresponds to a bacteria or preferably a yeast, which in its natural state presents a reduced tolerance to heavy metals, but when genetically transformed following this invention, with a vegetal-origin gene, presents high tolerance to heavy metals. Specifically, the cell in the invention is transformed with a vector that allows the expression of the AtHMA-1 gene of Arabidopsis thaliana, which codifies for a ATPase that may function as a heavy metal pump with great affinity in the intracellular space of the yeast, which confers this transformed cell a high capacity to remove heavy metals from aqueous solutions with high efficiency, so that this cell represents an improved alternative of great usefulness for biodepuration (bioremediation) of contaminated waters coming from industrial processes, and for the recovery of heavy metals intended for reutilization.

Recombinant cell transformed by an Arabidopsis thaliana ATPase AtHMA1 sequence that is useful to remove heavy metals from aqueous solutions; the use of said cell; a process to decontaminate a liquid medium from the presence of heavy metals.

### SUMMARY OF THE INVENTION

This invention corresponds to a cell, bacteria or preferably yeast, which in its natural state presents a reduced tolerance to heavy metals, but when genetically transformed—following this invention—with a vegetal-origin gene, presents high tolerance to heavy metals.

Specifically, we claim a cell that we transformed with a vector that allows the expression of the exogenous AtHMA-1 gene of Arabidopsis thaliana, which codifies for a ATPase and may function as a heavy metal pump with great affinity in the intracellular space of the yeast, which confers this transformed cell a high capacity to remove heavy metals, principally cadmium and copper, with high efficiency, from aqueous solutions. Thus, the invention that we present herein below comprises a transformed cell of great usefulness for the removal of heavy metals from liquid media, constituting an improved alternative for decontamination (bioremediation) of contaminated waters coming from industrial processes, and for the recovery of heavy metals intended for reutilization. The industrial application of this invention favors both the industrial reutilization of waters and/or heavy metals, and the decontamination of waters to be subsequently used in agriculture and cattle breeding.

### BACKGROUND INFORMATION EXISTING PRIOR TO THE DEVELOPMENT OF THIS INVENTION

The contamination of waters with heavy metals resulting from industrial productive activities is a reality in our own country and in several countries around the world. This configures a set of highly complex problems, which involves a vast array of strategic and economic aspects that have not been effectively resolved, contributing to the pauperization of a significant portion of available hydric resources. One of the main circumstances contributing to this situation, which frustrates the sanitary availability of public waters, is the nonexistence of an efficient and economically attractive method to decontaminate large water volumes overloaded with heavy metals. A highly efficient method would not only remove heavy metals, but it would, ideally, also recover heavy metals so that they may be reutilized.

It is frequently argued that the existing technical solutions to remove heavy metals such adsorption, inverse osmosis, ionic exchange, flocculation, among others, applied to liquid flows coming from activities such as mining, are not economically attractive in the short and medium term, so that it is indispensable to develop new technical alternatives allowing a high removal rate of heavy metals from aqueous media, in shorter times and with lower operating costs, in order to facilitate their early and massive application and to avoid endangering the availability of our hydric resources, mostly in those areas where they are most scarce.

In this sense, our invention provides a robust alternative with lower implementation costs that guarantees high efficiency in the removal of heavy metals from aqueous media. The invention we present here comprises the construction of a vector allowing the expression of a vegetal gene in a bacterial cell or preferably yeast. Specifically, the vector we designed permits, in the cell transformed by means of habitual genetic engineering techniques, the expression of the exogenous AtHMA-1 gene of Arabidopsis thaliana, which codifies for a ATPase that works as a high affinity heavy metal pump in the intracellular space, granting said transformed cell a great capability to incorporate heavy metals from the medium and to accumulate them in its interior (bioaccumulation, FIG. 1), while the cell transformed by the invention does not lose its capability to grow and multiply in said contaminated medium. This is, precisely, one of the technical advantages of this invention, because the transformed cell we claim herein not only possesses a high capability to bioaccumulate heavy metals, but does not lose its capability to duplicate its population. The advantage of multiplying itself at relatively constant rates favors reduction of the implementation cost because it facilitates the initiation of the decontamination process with a limited population of the cell transformed by the invention, which, as said population multiplies, also increases the quantity of heavy metals removed from the medium being treated.

As it will be understood by the individual experienced in the matter, the transformed cell in the invention represents a great innovation, useful in the preparation of industrial-level procedures for pre-treatment and bioaccumulation of heavy metals. The invention scope covers the generation of treatment services that may be specific for each industry through the adaptation of the transformed yeast in the invention to diverse growth conditions. Thus, it is possible to develop water decontamination procedures suitable for diverse industrial processes, depending on the heavy metal that must be removed and/or reutilized.

As we already mentioned, an important feature of our invention is associated with the opportunity to recycle heavy metals, which have an economic value and must be recovered from highly concentrated liquid waste products obtained, for instance, from mining activities. The preceding is perfectly feasible because, once the bioaccumulation procedure using the transformed cell in this invention is completed, the final biomass (final cellular population) may be subsequently processed with methodologies well known in the state of the art that include, among others, the recovery of biomass through precipitation or filtration; lysing of cells and recovery of the metals of interest with chromatographic techniques.

The use of the transformed yeast in this invention both for decontamination and/or recovery of heavy metals purposes as well as the related procedures are applicable at the national and international level, at companies that discharge effluents containing heavy metals, such as: mining companies, companies that launder salmon farming nets, cellulose plants, etc., all of which are very active in our country.

Since the hydric resource is one of the most important factors in the mining industry operating in the North of our country and, consequently, a source of conflict with the agronomic activities in valleys and canyons, the development of effective solution techniques is greatly needed so that the use of a system or procedure employing the cell in this invention may result in the reutilization of clean waters by the mining industry or farmers, which would bring economic and social benefits to both productive sectors.

Some alternatives to solve the problem of purifying waters contaminated with heavy metals have been brought up and executed at the scientific laboratory level, at a strictly experimental scale, with a series of strategies based on the capability of certain living organisms (bacteria and plants) to fix heavy metals in their biomass, thus removing them from the aqueous medium. The basic proposition in these systems, as an essential attraction element for their execution, has been the concept that heavy metal removal work, being done by a living organism, has a low operating cost potential. In fact, some bacteria and aquatic plants may grow in these contaminated streams and remove a significant portion of the heavy metals found in the aqueous medium. However, both bacteria and plants have maximum growth rates and maximum tolerances to ranges of environmental variables that significantly limit their potential utilization in industrial water treatment processes. The transformed cell in this invention solves these problems because, being a living organism, has a potentially low operating cost that, added to the genetic modification incorporated by us and described in detail herein below, possesses a greater capability to bioaccumulate heavy metals compared to the capability of organisms known in the state of the art.

One of the features of the transformed cell in this invention is its potential capability to decontaminate waters derived from industrial processes, facilitating the recovery of water suitable for reutilization by said industry or by farmers close to said industry, because the presence of waters contaminated with heavy metals is highly toxic to biological systems. In other words, an excessive presence of heavy metals such as copper or cadmium is a source of great toxicity for the fauna and flora that may be exposed to contaminated waters originated in an industrial process.

Copper, for example, being an essential trace element for every living being, in higher than normal concentrations becomes toxic for a large variety of cells. Copper is needed both by prokaryotic and eukaryotic cells, being required as a cofactor by a great variety of enzymes, participating in electron transport processes both at the mitochondrion and chloroplast level (Hall 2002; Clemens 2001; Himelblan and Amasino 2000). The redox nature of copper is crucial for its function; however, this very condition grants it the potential of causing oxidative damage when it is excessively present in the cell. Among the cellular damages caused by the hydroxyl radicals produced by the redox action of copper, we find the lipidic peroxidation of membranes, cleavage of the sugar-phosphate skeleton of nucleic acids and the denaturation of proteins (Schutzendubel and Polle, 2002; Berglund et al., 2002), all of which may provoke a cascade of events initially leading to cellular death and may terminate with the life of plants and animals exposed to highly dangerous copper concentrations.

Living organisms have developed a common copper homeostasis mechanism (when the same is found in normal concentrations), which consists in its transportation through the plasmatic membrane, joining it to chaperone proteins and distributing it in the diverse intracellular compartments, including its transport towards the secreting pathway for its elimination. in this last step, the participation of Cu²⁺ ATPases found in the Golgi apparatus of yeasts, humans and plants has been identified.

In plants, the cellular copper concentration regulation, detoxification and prevention of the copper-induced oxidative stress mechanism, has stages and elements similar to those described in some eukaryotic organisms (Sancenon et al., 2003; Mercer et al., 2003; Clemens 2001; Lin and Kossman 1990). The first step is the ion transport through the plasmatic membrane. In Arabidopsis thaliana, this stage is mediated by a high affinity transporter found in the plasmatic membrane, COPT 1, homologous to the CRT1 copper transporter of yeast (Kampfenkel et al., 1995). The second step is the sequestering of copper ions by chaperones, which distribute them towards the intracellular compartments where they are needed (Himelblan and Amasino, 2000). The third step in this regulation consists of transferring the ions from the chaperones to the transporters (generally Cu²⁺ ATPases pumps) found in the membranes of intracellular organelles. These transporters finally transport the ions towards the lumen of the organelles where they are stored and accumulated. Although the data provided by the Arabidopsis genome sequencing project indicate that there would be more than three genes that may codify for Cu²⁺ ATPases, only two have been described (Axelsen and Palmgren, 2001). These enzymes are: RANI, homologous to the Golgi Cu²⁺ ATPase of yeast, believed to be found in the vegetal Golgi and necessary for the ethylene signaling mechanism (Hirayama et al., 1999) and PAA1, a Cu²⁺ ATPase found in the chloroplast membrane, necessary to provide copper to the stroma enzymes and the lumen in the thylakoid (Shikanai et al. 2003).

A great worldwide effort, including our own work, has been undertaken to biochemically and genetically characterize the ATPases of plants. Precisely, a result of our research has been the characterization of ATPases in Arabidopsis thaliana, which provides the technical foundation for the invention that we propose and detail herein below.

It is necessary to mention that, prior to the development of this invention, it was known that both the ATPases of heavy metals and the Ca²⁺ ATPases belong to the P-ATPases super family (Axelsen and Palmgrem, 2001). The common characteristic of this P-ATPases super family is the presence of a phosphorylated intermediary during their catalytic cycle, the substrate specificity and effect of diverse inhibitors being the characteristic that differentiates the Ca²⁺ ATPases from the ATPases of heavy metals (Axelsen and Palmgren, 1998). However, up until the development of this invention, no description had been made of ion pumps of plants with dual activity, capable of transporting calcium and heavy metals.

Our studies suggested us, through in vitro metal transport tests, that one or more members of the P-ATPases super family of Arabidopsis thaliana would be capable of transporting calcium and heavy metals. In order to confirm the preceding, we designed active transport competition tests between copper and calcium in vegetal Golgi vesicles. While conducting these tests, we found that both ions (Ca²⁺ and Cu⁺) may be effectively transported and that, surprisingly, this transport activity is inhibited by thapsigargin, a specific inhibitor of Ca²⁺⁻ ATPases of the SERCA type (Ca²⁺⁻ ATPase of the sarcoplasmic reticle in animal cells). The fact that this putative Ca²⁺/Cu⁺ ATPase is inhibited by thapsigargin implies that it has a union domain for this kind of compound, which allowed us to obtain a fundamental tool to search in the Arabidopsis thaliana genome data base for the sequence(s) of gene(s) that codify for this protein. The results of our search indicated that the putative Ca²⁺/Cu⁺ ATPase is related to the ADNc At4937270 that codifies for the AtHMA1 (Arabidopsis thaliana Heavy Metal ATPase-1) heavy metals pump in Arabidopsis thaliana (Moreno, I. et al., 2008. AtHMA1 is a Thapsigargin-sensitive Ca²⁺/Heavy Metal Pump. J. Biol. Chem. 283: 9633-9641, incorporated to this patent for invention application as reference).

The enzyme that acts as a AtHMA1 heavy metals pump belongs to the ATPases Zn²⁺/Co²⁺/Cd²⁺/Pb²⁺ subclass Axelsen and Palmgrem, 2001) and it is the most divergent among the P_{IB}-ATPases of Arabidopsis thaliana. Previous (Higuchi et al., 2005) indicated that a mutant of Arabidopsis incapable of expressing AtHMA1 was sensitive to high concentrations of Zn and it was recently demonstrated that this enzyme is found in the chloroplast membrane. In addition, genic interruption mutants of Arabidopsis exhibit low copper content in the chloroplasts, suggesting a role of this enzyme in copper homeostasis in Arabidopsis.

Considering the above, we undertook to confirm whether AtHMA1 is capable of transporting both Ca²⁺ and heavy metals. Our studies (Moreno, I. et al., 2008) not only confirmed that AtHMA1 is an ATPase with an affinity with calcium and several heavy metals, but also allowed us to provide the foundations for the technological innovation that we detail herein below.

### DETAILED DESCRIPTION OF THE INVENTION

The purpose of our invention is a cell transformed with an exogenous gene that expresses a vegetal-origin protein, which provides said transformed cell with a significantly greater capability to grow and multiply at a high rate in aqueous media with high concentrations of heavy metals. In particular, our invention refers to a bacterium or preferably a transformed yeast with the AtHMA-1 gene of Arabidopsis thaliana, which grants the transformed cell a greater capability to grow in media with high concentrations of heavy metals, to remove said heavy metals from the medium and to intracellularly accumulate them (bioaccumulation, FIG. 1). Thus, when the transformed cell in the invention is placed in contact with an aqueous medium with high concentrations of heavy metals, it facilitates the decontamination of said medium and provides, in addition, an alternative for the recovery of said heavy metals. The scope of this invention allows a potential application for the effective decontamination at a low operating cost of, for example, waters contaminated by industrial works such as mining, where the transformed cell in this invention can help to decontaminate and/or recycle diverse metals such as Cu, Cd, Co, Ca, Zn and Mn.

Our invention is based on the fact that we have confirmed that when incorporating the AthHMA1 gene of Arabidopsis thaliana into yeasts using molecular genetics techniques, said gene is capable of over-expressing itself in the transformed yeast permitting a greater active and selective transport of metallic cations from the extracellular medium towards the interior of the cell, where they are isolated and fixed in the vacuole (bioaccumulation), in this way preventing them from exercising their toxic action on the cell and allowing the unfettered growth of the yeast population, removing more heavy metals with the increased collective biomass of the yeast population.

Thus, we have built a recombinant yeast (transformed with an exogenous gene), which shows a great capability to accumulate heavy metals, becoming a great alternative for the decontamination of waters and, in addition, for the recovery of said metals. The metals' concentrations in which the transformed yeast of this invention is capable of growing and accumulating them, are far superior to those reported for other microorganisms and plants that have been offered and researched to be used for these purposes. As an example, a tolerance of up to 3 mg/l of Copper contaminant is reported for bacteria, and up to 1 mg/l for plants, while in respect of the transformed yeast in this invention, this yeast has grown and multiplied without problems in concentrations above 200 mg/l of the same metal, removing and accumulating in its biomass more than 80% of the copper found in the medium.

In this way, the transformed yeast in this invention represents a robust alternative to be used in the mining industry, the recycling of heavy metals and industrial utilization waters and in agriculture, in the ecological field and in the control of contaminant agents because it can be used in:

Processes to bioremediate natural sources of waters contaminated with heavy metals such as copper and cadmium, where said sources may be ponds, lakes, rivers, etc.

Processes to obtain copper in the mining industry.

Treatment of liquid industrial waste.

Reutilization of heavy metals such as copper and cadmium, removed from contaminated waters.

Cleanup of waters for their reutilization in the mining industry or agricultural production.

In particular, this invention may be of immense usefulness in the mining industry, because, through the use of bioreactors, the mining industry could reuse a high percentage of the copper wasted in aqueous residues. In addition, the decontaminated water could be used once again in mining processes or agriculture.

As already mentioned, the transformed yeast in this invention may also be used in decontamination and recovery applicable, for example, to cadmium (one of the most abundant contaminating metals resulting from diverse productive activities, mining and agriculture among them) and applicable to several other metals. The functional characterization of the transformed yeast in this invention shows, as in FIG. 2, that it may be used in the accumulation of other metals such as: Co, Ca, Zn and Mn.

In respect of the conventional systems now in use, systems based on dead or living biomass (phytoremediation, biosorption), this invention comprises several advantages both in its application and in the efficiency of metal removal from the contaminated medium, which ultimately translates into a cost-benefit ratio favorable to the invention we present herein. As an example, and to underline the advantages of this application in respect of what is already known in the state of the art, in Table 1 we present a comparison among the diverse systems in the market, identifying their economic and technical advantages and disadvantages.

TABLE 1
Comparison of systems for the removal of metals from
industrial residues and/or aqueous solutions. Includes the most studied and
used physicochemical and bioremediation methods. These, albeit effective,
present several disadvantages such as important costs in energy and/or
chemical products consumption terms. A clear example is chemical
precipitation, which, although it effectively eliminates heavy metals, it
creates a new environmental problem: the muds that must be subsequently
stored (Source: United States Environmental Protection Agency, EPA).

| Methods | Advantages | Disadvantages |
|---|---|---|
| Precipitation, | System simplicity. | Presence of organic |
| using chemicals or | High level of heavy | agents diminishes its |
| systems based on | metal removal. | effectiveness. |
| bacterial | Low operating cost. | Coagulant and |
| metabolism. | | flocculant agents are |
| | | needed to separate |
| | | metals from the effluent. |
| | | Generation of muds |
| | | with a high treatment |
| | | cost. |
| Ionic Exchange | Ions may be eliminated | Presence of Calcium, |
| | at a very low | Sodium and Magnesium |
| | concentration. | diminishes its yield |
| | High selectivity. | because the may |
| | Metals may be | saturate the resin. |
| | recovered via | Possible competition |
| | electrolysis. | between heavy metals |
| | | and other cations. |
| | | Resins do not tolerate |
| | | pH variations. |
| | | The organic materials |
| | | present in the solutions |
| | | may decompose resins. |
| | | The contaminated |
| | | solution must be |
| | | previously treated to |
| | | eliminate materials in |
| | | suspension. |
| Adsorption | Highly effective at very | The cost of the |
| | low metal concentrations. | adsorbent and its |
| | Easy to operate. | regeneration may be very |
| | Permits metal fixation in | high. |
| | the presence of other | The adsorption capacity |
| | cations. | is highly dependent on |
| | It is possible to recover | pH. |
| | heavy metals. | It is necessary to |
| | The adsorbent may be | eliminate the matter in |
| | regenerated. | suspension before the |
| | | effluent is treated. |
| Inverse Osmosis | Presents high removal | Medium selectivity and |
| | capability. | tolerance to pH changes. |
| | Automated process. | Short half life when |
| | Does not provoke | used with corrosive |
| | changes in the chemical | solutions. |
| | composition of residual | Requires the generation |
| | waters. | of high pressures for |
| | It is possible to recover | proper operation. |
| | heavy metals. | High membrane |
| | | replacement cost. |
| | | It is necessary to |
| | | separate the insoluble or |
| | | suspended parts to |
| | | prevent membrane |
| | | saturation. |
| Bioadsorption | Capability to treat large | Costly recovery of |
| | water volumes because | adsorbed metals |
| | of process speed. | because of the poor |
| | Capability to manipulate | efficiency of the final |
| | several heavy metals and | separation of the effluent |
| | residue mixtures. | and the biomass. |
| | Low investment. | Operates optimally in |
| | Operates within an | diluted solutions (≈3 mg |
| | ample range of | metal/L) |
| | physicochemical | Depletion of the |
| | conditions including | bioadsorbent matter. |
| | temperature, pH and the | |
| | presence of ions. | |
| Phytoremediation | Environmentally friendly | Method not too |
| | method, allowing the use | selective. |
| | of vegetal species that | It is not possible to |
| | naturally accumulate | separate heavy metals |
| | heavy metals. | from the organic matter |
| | Allows processing large | for their reutilization. |
| | volumes of contaminated | High implementation |
| | waters. | and maintenance costs. |
| | | Sensitive to variations |
| | | in the physicochemical |
| | | conditions of the effluent |
| | | (pH, salinity, etc.). |
| Bioaccumulation, | Capability to treat | It is necessary to |
| using the | large water volumes | maintain a bioreactor. |
| transformed | because of the process | It is necessary to |
| yeast in this | speed. | supply the system with |
| invention. | Capability to | nutrients to enable the |
| | manipulate several | subsistence of the |
| | heavy metals and | organism used by the |
| | residue mixtures. | bioreactor (ex. Source |
| | Simplicity of | of carbon, nitrogen, |
| | establishing the range | etc.). |
| | of physicochemical | |
| | conditions, including | |
| | temperature, pH and | |
| | presence of ions. | |
| | Since it involves living | |
| | organisms that keep | |
| | living and multiplying | |
| | during the process, it | |
| | practically supplies | |
| | itself with the | |
| | bioaccumulator | |
| | element. | |
| | It is possible to | |
| | recover the | |
| | accumulated metals. | |
| | Operates both in | |
| | diluted and | |
| | concentrated solutions | |
| | (from 3 mg/l up to | |
| | 200 mg/l and above). | |
| | Low investment. The | |
| | use of facultative | |
| | anaerobic organisms | |
| | (ex. yeasts) brings | |
| | down implementation | |
| | and maintenance costs. | |

### EXEMPLIFICATION OF THE INVENTION

Herein below we describe examples that allow the reproduction of this invention and the results obtained by us that demonstrate the great advantage of transformed yeast in this invention to bioaccumulate metals. These examples only intend to illustrate the invention without limiting it because the individual who is knowledgeable of the state of the art will notice that it is possible to extend its usefulness. We demonstrate here the potential of the transformed yeast in this invention both to depurate contaminated waters and to promote the recovery of industrially interesting metals.

### DESCRIPTION OF FIGURES

FIG. 1. Model of metal bioaccumulation. It is based on the absorption of metallic specimens through accumulation mechanisms operating at the interior of living biomass cells.

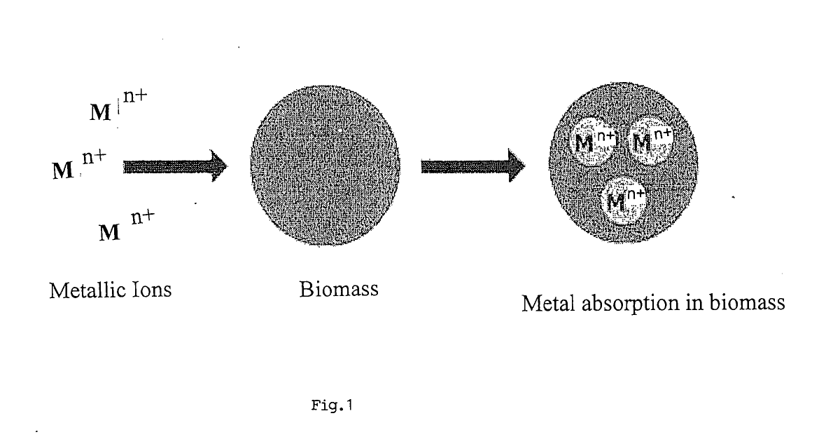

FIG. 2.—Relative activity of the accumulation of diverse metals in the transformed yeast in this invention that over expresses a heavy metals pump of vegetal origin, compared with native yeast, without any transformation. The figure presents a graph of the relative accumulation activity (expressed as a % normalized to the maximum measured activity). The black bars represent the accumulation capacity for cadmium, cobalt, copper, manganese, calcium and zinc in yeasts transformed with the heavy metals pump, while the white bars represent the same activity but in the yeast without any transformation.

FIG. 3.—This figure shows that AtHMA1 functionally complements the Δycf1 hypersensitive to cadmium mutant. The functional complementation of Δycf1 by AtHMA1 was tested in a solid medium. (Δycf1-pGPD426) is the transformed yeast with vector not containing the ADNc that codifies for AtHMA1 and (Δycf1-AtHMA1) is the yeast transformed with the vector containing the ADNc that codifies for AtHMA1. Cells taken from both strains were cultured in growth media containing 70 μM of CdCl₂. The DTY165 wild strain transformed with vector not containing the ADNc that codifies for AtHMA1 (WT-pGPD426) was used as control.

FIG. 4.—AtHMA1 increases the tolerance to cadmium of the wild yeast strain (W303). The figure shows the growth curves of (A) WT (W303), wild-type yeast transformed with the empty vector (pGPD426) and (B) WT (W303), wild yeast transformed with the vector containing the ADNc that codifies for AtHMA1. The yeast cells were grown at 30° C. in a liquid medium supplemented with 0 μM; ▴:70 μM; ▾:100 μM; ♦:150 μM; and : 200 μM CdCl₂. The cellular densities (OD at 600 nm) were determined during 5 days. The tests were made in triplicate and the values represent±the Standard Deviation.

### EXAMPLES

All the preparation stages for the ADNc At4g37270 to be inserted into the expression vector, as well as the transformation of bacteria and yeasts with said vector in addition to the functional and bioaccumulation studies of the transformed yeast, are described in Moreno, I. et al., (2008), which is herein incorporated as reference.

### Example 1

Cloning and molecular identification of the gene that codifies for a calcium and heavy metals ATPase of Arabidopsis thaliana, useful to transform the cell—bacteria or yeast—in this invention and in this way grant the transformed cell a great capability to accumulate heavy metals.

The cloning of ADNc At4g37270 was made following protocols well known in the state of the art and described in Sambrook, J. et al., (1989).

The ADNc At4g37270 (Nucleotidic sequence No. 1) was amplified by PCR from an ADNc gene library of Arabidopsis thaliana using the AccuTherm™ (GeneCraft) DNA-polymerase and starters that flank the codifying region of At4g37270. The sequences of the starters used are: 5′-CGCTTGA GATCTAATTCGTCGACCATGGAA-3′ (sense strand starter; the BgIII restriction site is underlined) and 5′-AGACAAGCGGCCGCAAGTTACCCCCTAATG-3′ (counter sense starter; the NotI restriction site is underlined). After verifying it through sequentiation, the product of PCR was cut with BgIII and NotI in order to be bound to the pGPD426 expression vector in yeast previously digested with BamHIINotI, in this way obtaining the pGPD426-AtHMA1 expression vector, which was used to transform strains of DH5α Escherichia coli in order to amplify the vector and keep a stock of the same.

### Example 2

### Yeast Strains

The following yeast strains of S. cerevisiae were used in the development of this invention:

YR98 [MATα, ade2 his3-Δ200 Ieu2-3,112 lys2-Δ 201 ura3-52] and its isogenic mutant Δpmr1 (YR122) [pmr1-P1::Δ1:: Leu2];
W303 [MATα, ade2-1 can1-100 his3-11, 15 leu2-3, 112 trp1-1 ura3-1□ and its isogenic mutant K616 [pmr1::His3 cnb1::Leu2 pmc1:Trp1□;
DTY165 [MATα, ura3-52 leu2-3, 112 his3-Δ200 trp1-Δ901 lys2-801 suc2-Δ9] and its isogenic mutant Δycf1 (DTY167) (ycf1::HisG).

The S. cerevisiae yeast strains were grown at 30° C. in YPD medium.

### Example 3

### Transformation of Yeasts with the pGPD426-AtHMA1 Expression Vector

The transformation of yeasts was carried out with the LiAc method (Dietz, D., et al., 1992). After the transformation, the cells were grown in a selective medium (0.67% nitrogenated base of yeast without amino acids, 2% glucose) and supplemented with the appropriate auxotrophic requirements. The transformers were tracked by means of selection in minimum media lacking uracil. In this medium, the nontransformed mutant cells die because they do not have the genes needed to synthesize uracil (i.e. they are ura-). On the contrary, the cells transformed with the pGPD426 vector (that carries the genes required to synthesize uracil) are capable of growing in this medium (i.e. the convert into URA+ cells).

### Example 4

### Expression of AtHMA1 in Yeasts Transformed with the pGPD426-AtHMA1 Expression Vector

The expression of AtHMA1 in the transformed yeast cells was verified using RT-PCR. The transformed yeast cells were grown in a selective medium up to O.D (600 nm) of 0.6. The cells were collected through centrifugation and total ARN was extracted from them with the Chomczynski phenol-chloroform extraction method (Chomczynski, P., Sacchi, N. 1987). The ADNc was prepared from the isolated ARN. For this purpose we used the “RevertAid™” First Strand cDNA Synthesis (from Ferrmentas) kit with Oligo (dT) and 1 μg of total RNA. After the reverse transcription of the ARNm to ADNc, a fragment of 565 by of the ADNc from AtHMA1 was amplified by PCR using the following starters: 5′-ATGATGTTAACTGGGGACC-3′ (sense strand starter) and 5′-TAATGTGCAGAGCTTAAACTGTTGCTGCTGCTACT-3′ (counter-sense starter). The amplification of the codifying ADNc for ACT1 (actin 1, house-keeping) was used as internal control in all reactions (Del Aguila et al., (2005). The products of PCR were separated with electrophoresis in agarose gel.

### Example 5

### Tests of Toxicity Caused by Metals

### Example 5.1

Tests of toxicity caused by cadmium in the Δycf1 strain transformed with pGPD426-AtHMA1 versus cells of the same strain transformed with the empty vector.

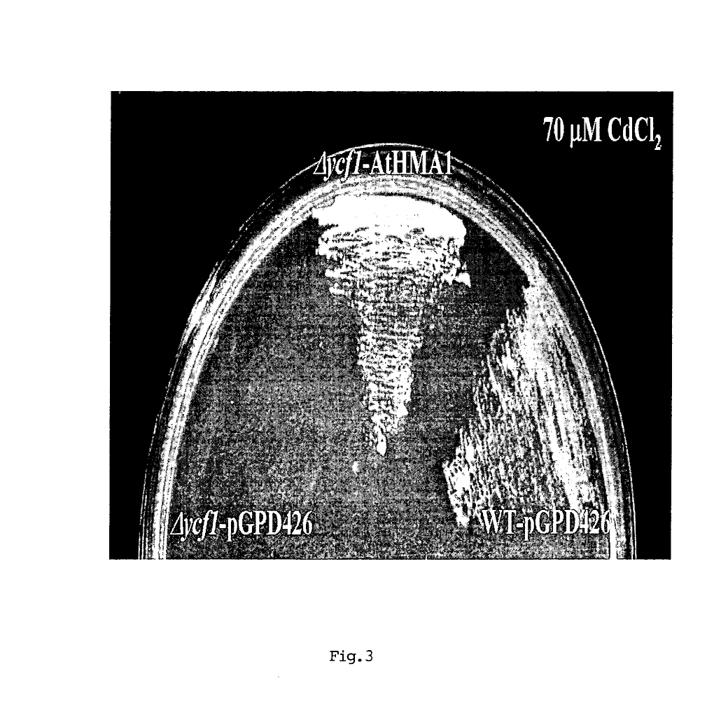

This test uses cells of Δycf1 mutant yeast, which is hypersensitive to cadmium, so that in order to evaluate changes in sensitivity we tested Δycf1 yeast cells transformed with pGPD426-AtHMA1 (or with the empty vector, pGPD426), which were grown in a solid growth medium supplemented with 70 μM Cd²⁺ and evaluated the sensitivity to Cadmium. Those transformed yeast cells in the invention that express the AtHMA1 enzyme of Arabidopsis thaliana grew in a way similar to wild yeast cells (FIG. 3). While, as it could be expected, the Δycf1 mutant strain transformed with the empty pGPD426 vector did not survive in this medium containing cadmium (Antebi, A., and Fink, G. (1992).

The high tolerance reached by the Δycf1 yeast cells transformed in accordance with our invention is also confirmed when growing transformed cells in a liquid growth medium supplemented with increasing concentrations of this heavy metal. The first remarkable effect of the expression of AtHMA1 in Δycf1 cells, is a longer generation time or Gt (corresponding to the duplication time of each population of yeast cells) under control conditions compared with the growth shown by Δycf1 and the wild strain (DTY165). At greater Cadmium concentrations (from 100 to 200 μM Cd²⁺), the Δycf1 cells that express AtHMA1 following the invention described herein grow faster than the control strain. The preceding is reflected in a shorter duplication time compared to the control strain (see Table 2).

Table 2. Generation Times (Gt) of S. cerevisiae strains transformed with the empty pGPD426 vector or the same vector but operationally bound to the ADNc of AtHMA1. The Generation Times (Gt) were measured during the exponential growth phase on selective media supplemented with increasing concentrations of CdCl₂. The exponential growth rate of the yeast cultures is expressed as the generation time in hours (h), that is, the duplication time of each population of tested yeasts.

TABLE 2

| | Generational time (h) | | | | |
|---|---|---|---|---|---|
| | | | 100 μM | 150 μM | 200 μM |
| | 0 μM Cd | 70 μM Cd | Cd | Cd | Cd |
| WT- | 4.227 | 9.476 | 38.983 | 101.610 | 89.460 |
| pGPD426 | (±0.077) | (±1.737) | (±3.654) | (±9.140) | (±22.410) |
| Δycf1- | 4.776 | 69.765 | 75.760 | 313.400 | 336.550 |
| pGPD426 | (±0.083) | (±0.225) | (±0.550) | (±8.100) | (±26.950) |
| Δycf1- | 12.613 | 15.313 | 13.767 | 14.467 | 14.193 |
| AtHMA1 | (±0.421) | (±0.841) | (±0.152) | (±1.081) | (±0.979) |

Values are averages of triplicate tests ±ES.

In order to confirm that the high tolerance to Cadmium reached by the transformed Δycf1 yeast cells in accordance with our invention is owed to the expression of AtHMA1, we tested the capability of this heavy metal to activate this ATPase present in fractions of isolated microsomal membranes, as described in Moreno et al., (2008). Our results indicate that the fractions of isolated microsomal membranes from the Δycf1 yeast cells transformed following our invention present an ATPase activity that is stimulated six times more than the one present in Δycf1 yeast cells only transformed with the empty vector (Moreno et al., (2008), demonstrating the high intracellular activity and direct participation of this exogenous gene in the high tolerance to Cadmium reached by the transformed Δycf1 yeast cells, promoting the removal of cadmium from the citosol by means of its active transport for its accumulation in cellular compartments (bioaccumulation).

### Example 5.2

Tests of toxicity caused by cadmium in W303wild yeast cells transformed with pGPD426-AtHMA1 versus cells of the same strain transformed with the empty vector.

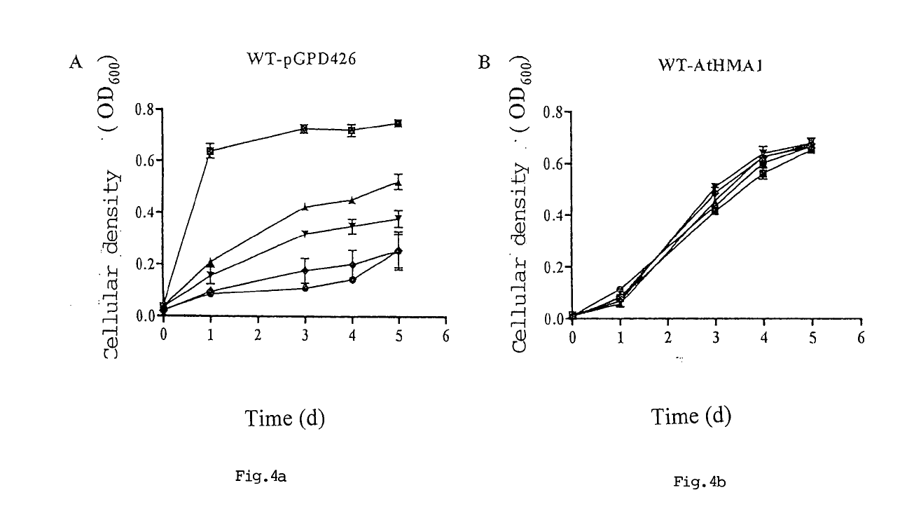

In order to determine if AtHMA1 is also capable of increasing the tolerance to cadmium of the wild strain, we compared the growth in YPD medium supplemented with high concentrations of Cd²⁺ during 5 days in wild cells (W303) transformed with the empty vector (pGPD426) and the same wild cells but transformed with the vector that allows the expression of AtHMA1 of Arabidopsis thaliana. The wild strain cells transformed with the empty vector (WT-pGPD426) grow in all the tested concentrations of Cd²⁺ (from 70 to 200 μM) (FIG. 4), however, the duplication time increases with higher concentrations of Cd²⁺ (see Table 3).

With higher cadmium concentration, the wild strain cells that express the introduced AtHMA1 gene (WT-AtHMA1 cells) grew faster than the wild strain cells to which the vector without the AtHMA1 gene was introduced (WT-pGPD426 cells), while we did not observe a great reduction in duplication time with increasing cadmium concentration (see Table 3).

The results clearly demonstrate that the expression of AtHMA1 reverts the Cadmium hypersensitivity phenotype of the Δycf1 strain, while it grants to the wild strain transformed in order to over express AtHMA1 a notably greater tolerance to this heavy metal.

Table 3. Generation Times (Gt) of (W303) S. cerevisiae strains transformed with the empty pGPD426 vector or the same vector but operationally bound to the cADN of AtHMA1. The Generation Times (Gt) were measured during the exponential growth phase on selective media supplemented with increasing concentrations of CdCl₂. The exponential growth rate of the yeast cultures is expressed as the generation time in hours (h), that is, the duplication time of each population of tested yeasts.

TABLE 3

| | Generational time (h) | | | | |
|---|---|---|---|---|---|
| | | | 100 μM | 150 μM | 200 μM |
| | 0 μM Cd | 70 μM Cd | Cd | Cd | Cd |
| WT- | 5.764 | 9.633 | 60.640 | 458.517 | 631.665 |
| pGPD426 | (±0.640) | (±1.185) | (±5.043) | (±16.097) | (±27.036) |
| WT- | 30.097 | 28.093 | 15.660 | 17.280 | 31.213 |
| AtHMA1 | (±0.048) | (±0.664) | (±1.983) | (±0.592) | (±0.292) |

Values are averages of triplicate tests ±ES

### Example 5.3

Tests of toxicity caused by other heavy metals in wild Δycf1 yeast cells transformed with pGPD426-AtHMA1 versus cells of the same strain transformed with the empty vector.

The sensitivity to other transition metals was tested growing transformed Δycf1 cells in a solid growth medium supplemented with 6 mM CoCl₂, 4 mM CuSO₄ or 28 mM ZnCl₂. The test plates holding the yeasts were incubated for 5 days at 30° C.

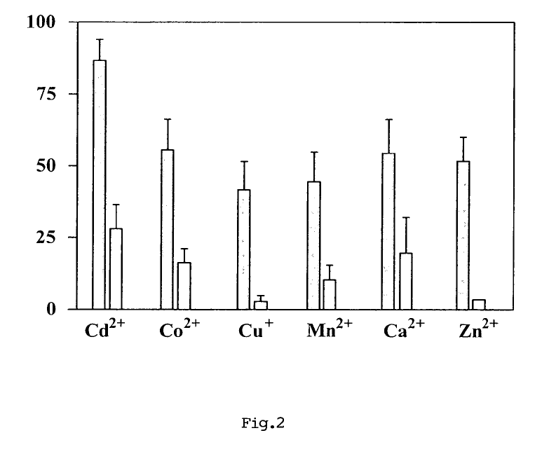

FIG. 2 shows that Δycf1 cells of the mutant strain transformed with the empty vector (Δycf1-pGPD426, white bars) exhibit poor growth in a solid medium containing 6 mM CoCl₂, 4 mM CuSO₄ or 28 mM ZnCl₂. However, the cells that express AtHMA1 (Δycf1-AtHMA1, black bars) grow normally under the same conditions.

In order to confirm that the higher tolerance to these metals observed in the transformed Δycf1 yeast cells in accordance with our invention is owed to the expression of AtHMA1, we tested the capability of these heavy metals to activate this ATPase present in fractions of isolated microsomal membranes, as described in Moreno et al., (2008). Our results indicate that the fractions of isolated microsomal membranes from the Δycf1 yeast cells transformed following our invention present an ATPase activity that is stimulated 15 times more in the presence of Zn²⁺; 13 times more in the presence of Cu⁺ and 3 times more in the presence of Co²⁺, than the ATPase activity measured in fractions of isolated microsomal membranes from the Δycf1 yeast cells transformed only with the empty vector (Moreno et al., (2008), which confirms the high intracellular activity and direct participation of this exogenous gene in the greater tolerance to these metals reached by the transformed cells (Δycf1-AtHMA1), promoting the removal of these metals from the citosol by means of active transport for their accumulation in cellular compartments (bioaccumulation).

cDNA At4g37270 of the heavy metal ATPase used
for the stable transformation of yeast:
sense strand
2460 nucleotides
Nucleotidic sequence No. 1
ATGGAACCTGCAACTCTTACTCGTTCTTCCTCTCTTACTAGATTCCCTT
ATCGTCGTGGTTTATCCACTCTCCGACTCGCTCGAGTCAACTCGTTCTC
AATTCTTCCACCTAAAACTCTTCTCCGTCAAAAACCGCTTCGTATCTCT
GCTTCCCTTAGTCTTCCACCACGGTCGATTCGTCTACGTGCTGTCGAAG
ATCACCATCACGATCATCATCACGATGACGAGCAAGATCATCACAACCA
CCATCATCATCACCATCAACACGGATGCTGTTCTGTGGAATTGAAAGCG
GAGAGTAAGCCTCAGAAGGTGTTGTTCGGATTCGCTAAAGCTATCGGAT
GGGTTAGATTGGCCAATTACCTCAGAGAGCATCTTCATCTTTGCTGCTC
CGCCGCTGCAATGTTCCTCGCTGCCGCCGTCTGTCCTTACCTTGCTCCT
GAACCTTACATTAAGTCTCTTCAGAACGCATTCATGATTGTTGGTTTTC
CTCTTGTTGGAGTTTCAGCATCTCTCGACGCACTTATGGATATAGCTGG
AGGAAAAGTGAACATCCATGTCTTGATGGCACTTGCGGCTTTTGCATCT
GTGTTTATGGGAAATGCTTTGGAAGGAGGATTGCTTCTAGCTATGTTCA
ATCTTGCTCATATTGCTGAGGAGTTCTTTACTAGTCGATCAATGGTGGA
TGTCAAAGAATTGAAAGAGAGTAATCCAGATTCTGCATTGTTGATCGAA
GTACACAATGGCAATGTTCCAAATATATCTGATTTGTCATACAAAAGCG
TTCCTGTGCACAGCGTAGAAGTTGGATCCTATGTTTTGGTTGGAACTGG
TGAGATTGTGCCTGTAGATTGCGAAGTCTATCAAGGTAGTGCTACAATT
ACAATTGAGCACTTGACTGGGGAAGTCAAGCCGTTGGAGGCAAAAGCTG
GAGATAGAGTGCCTGGTGGTGCAAGAAATTTGGATGGCAGAATGATTGT
AAAGGCTACAAAGGCATGGAATGATTCGACGCTTAACAAGATTGTACAG
CTGACCGAGGAAGCACATTCTAATAAACCCAAACTTCAGAGATGGCTGG
ATGAGTTTGGCGAGAATTACAGCAAGGTTGTCGTTGTTTTGTCACTTGC
AATTGCCTTCCTAGGTCCATTTTTGTTCAAGTGGCCTTTTCTCAGCACC
GCAGCATGTAGAGGATCTGTTTACAGAGCATTGGGACTTATGGTGGCCG
CATCACCATGTGCTCTGGCCGTAGCTCCATTGGCTTATGCTACTGCTAT
TAGTTCCTGTGCAAGAAAGGGAATATTGCTGAAAGGTGCACAGGTTCTA
GATGCTCTTGCGTCTTGCCATACTATTGCTTTTGACAAAACTGGTACCT
TAACAACCGGCGGCCTTACTTGTAAAGCAATTGAACCCATTTATGGGCA
CCAAGGAGGAACTAATTCAAGTGTAATAACTTGCTGCATTCCAAATTGT
GAAAAAGAAGCTCTTGCAGTTGCGGCTGCCATGGAGAAGGGCACCACGC
ATCCTATTGGAAGAGCTGTTGTAGATCACAGTGTGGGTAAGGATCTTCC
TTCTATTTTTGTTGAAAGCTTCGAATATTTTCCTGGTAGAGGCCTTACT
GCTACTGTCAACGGTGTTAAGACAGTAGCTGAAGAGAGTAGATTACGAA
AAGCATCACTTGGTTCTATAGAGTTCATTACCTCACTTTTCAAATCTGA
AGATGAATCTAAACAGATCAAGGATGCTGTAAACGCGTCTTCGTACGGA
AAGGACTTCGTTCATGCTGCTCTTTCTGTTGATCAAAAGGTAACATTGA
TTCACCTCGAAGATCAGCCTCGTCCAGGGGTGTCAGGAGTTATAGCAGA
ACTTAAAAGCTGGGCCAGACTCCGAGTAATGATGTTAACTGGGGACCAT
GATTCAAGTGCTTGGAGAGTTGCAAACGCAGTGGGTATTACCGAAGTCT
ACTGCAACCTAAAGTCAGAGGATAAGTTAAATCATGTAAAGAACATTGC
TCGGGAAGCAGGTGGAGGTTTAATTATGGTAGGAGAAGGGATTAATGAT
GCTCCAGCTCTAGCAGCTGCAACAGTGGGGATTGTTCTTGCTCAACGAG
CGAGTGCCACTGCGATTGCCGTTGCTGACATCTTACTGCTTCGAGACAA
CATCACCGGTGTTCCGTTCTGTGTCGCTAAATCCCGCCAGACAACATCA
TTGGTCAAGCAAAACGTAGCTCTTGCATTAACATCGATATTCTTGGCCG
CTCTTCCTTCAGTTTTAGGGTTTGTCCCATTGTGGTTGACGGTACTTCT
ACATGAAGGCGGGACTCTTCTGGTGTGTCTAAACTCAGTACGTGGTCTA
AACGATCCATCATGGTCGTGGAAACAAGACATAGTTCATCTAATCAACA
AGTTACGCTCACAAGAACCAACCAGTAGCAGCAGCAACAGTTTAAGCTC
TGCACATTAG

### LIST OF REFERENCES

Antebi, A., and Fink, G. (1992). The Yeast Ca²⁺-ATPase Homologue, PMR1, is Required for Normal Golgi Function and Localizes in a Novel Golgi-like Distribution. Mal. Biol. Cell, 3: 633-654.
Axelsen, K. B. and Palmgren, M. G. (1998), Evolution of substrate specificities in the P—type ATPase superfamily. J. Mol. Eval. 46: 84-101.
Axelsen, K. B. and Palmgren, M. G. (2001). Inventory of the Superfamily of P—Type Ion Pumps in Arabidopsis. Plant Physiol. 126: 696-706.
Del Aguila, E. M., Dutra, M. B., Silva, J. T., Paschoalin, V. M. F. (2005). Comparing protocols for preparation of DNA-free total yeast RNA suitable for RT-PCR. BMC Mal. Biol. 6: 9.
Gietz, D., et al., (1992). Improved method for high efficiency transformation of intact yeast cells. Nucleic. Acids Res. 20: 1425.
Higuchi, M., Sonoike, K. (2005). A disruption mutant of hma1, a putative metal transporter, in Arabidopsis thaliana shows sensitivity to high concentration of Zn and altered kinetics of nonphotochemical quenching of chlorophyll fluorescence. In Photosynthesis: Fundamental Aspects to Global Perspectives, A. van der Est and D. Bruce (eds), pp. 716-718. International Society of Photosynthesis.
Moreno, I. et al., (2008). AtHMA1 Is a Thapsigargin-sensitive Ca²⁺/Heavy Metal Pump, J. Biol. Chem. 283: 9633-9641.
Sambrook, J., Fritsch, E. F., Maniatis, T. (1989), Molecular Cloning: A Laboratory Manual, 2nd Ed., Cold Spring Harbor Laboratory, Cold Spring Harbor, N.Y.
Chomczynski, P., Sacchi, N. (1987). Single step method of RNA isolation by acid guanidinium thiocyanatephenolchloroform extraction. Anal. Biochem. 162: 156-159.

1. A recombinant cell, transformed with an exogenous gene, useful to remove contaminants from aqueous sources CHARACTERIZED because said transformed cell expresses the nucleotidic sequence that codifies for the AtHMA-1 ATPase of Arabidopsis thaliana (SEQ ID NO: 5), and where said cell, once transformed, is capable of removing, with high efficiency, heavy metals from aqueous media and accumulating them in its interior (biomass).

2. The cell in claim 1 CHARACTERIZED because it is a eukaryotic cell.

3. The cell in claim 2 CHARACTERIZED because it is a yeast.

4. The cell in claim 1 CHARACTERIZED because it is a bacteria.

5. The use of the transformed cell according to claim 3 CHARACTERIZED because said cell is useful to decontaminate an aqueous medium contaminated with at least one heavy metals, which involves the incorporation of said cell into said contaminated aqueous medium, where the transformed cell multiplies itself while progressively incorporating and accumulating said at least one heavy metal in its biomass, thus removing said at least one heavy metal from the contaminated medium.

6. The use of the cell according to claim 5 CHARACTERIZED because said cell is useful to decontaminate aqueous media contaminated with heavy metals, resulting from industrial processes.

7. The use of the cell according to claim 6 CHARACTERIZED because said cell is useful to decontaminate aqueous media contaminated with heavy metals, resulting from mining processes.

8. The use of the cell according to claim 7 wherein said at least one heavy metal comprises one or more of the metals selected from the group consisting of copper, cadmium and zinc.

9-10. (canceled)

11. The use of the transformed cell according to claim 3 CHARACTERIZED because said cell is useful to recover at least one heavy metal from an aqueous medium that contains heavy metals, which involves the incorporation of said cell into said aqueous medium, where the transformed cell multiplies itself while progressively incorporating and accumulating said at least one heavy metal in its biomass in such a way that said at least one heavy metal may be subsequently recovered from the recovery of the biomass of said transformed cell.

12. The use of the cell according to claim 11 wherein said at least one heavy metal comprises one or more of the metals selected from the group consisting of copper, cadmium, zinc, cobalt and manganese.

13-16. (canceled)

17. Procedure to decontaminate a liquid medium from the presence of a heavy metal CHARACTERIZED because it comprises the steps of
a) placing said medium to be decontaminated at a temperature between 27° C. and 33° C.
b) incorporating the transformed cell of claim 3 into the medium to be decontaminated described in step a)
c) allowing the multiplication of said cell in said medium, until the cellular density reaches an O.D (600 nm) of 0.75 to 1.0 and
d) removing the cells through precipitation or filtration.

18. The procedure in claim 17 CHARACTERIZED in that said heavy metal corn a rises one or more of the metals selected from the grow consisting of copper, cadmium and zinc.

19-20. (canceled)

21. Procedure to recover a heavy metal from a liquid medium CHARACTERIZED because it comprises the steps of
a) placing said medium to be decontaminated at a temperature between 27° C. and 33° C.
b) incorporating the transformed cell of claim 3 into the medium to be decontaminated described in step a)
c) allowing the multiplication of said cell in said medium, until the cellular density reaches an O.D (600 nm) of 0.75 to 1.0
d) collecting the cells through precipitation or filtration and
e) lysing the cells and extracting the heavy metal through chromatography.

22. The procedure in claim 21 CHARACTERIZED in that said heavy metal recovered from said medium comprises one or more of the metals selected from the groom consisting of copper, cadmium, zinc, cobalt, manganese and calcium.

23-27. (canceled)
